(19) United States
(12) Patent Application Publication
(10) Pub. No.: US 2026/0335660 A1
(43) Pub. Date: Oct. 29, 2026

(54) Electrical Energy Storage Device for an Implantable Medical Stimulation Device

(71) Applicant: BIOTRONIK SE & CO. KG, Berlin (DE)

(72) Inventors: Ingo WEISS, Pforzheim (DE); Thomas DOERR, Berlin (DE)

(73) Assignee: BIOTRONIK SE & CO. KG, Berlin (DE)

(21) Appl. No.: 19/112,441

(22) PCT Filed: Aug. 28, 2023
(86) PCT No.: PCT/EP2023/073565

§ 371 Date: Mar. 17, 2025

(30) Foreign Application Priority Data
Sep. 30, 2022 (EP) 22198947.8

(51) Int. Cl. A61N 1/378; H01M 4/70

(52) U.S. Cl. CPC A61N 1/378; H01M 4/70; H01M 2220/30

(57) ABSTRACT

Electrical energy storage device for an implantable medical stimulation device including a housing, first and second electrical electrodes having different electrical polarities, one of the first and second electrical electrodes being formed by an electrode element having an electrically conductive electrode body arranged in the housing, and a current collector arrangement electrically connected to the electrode element. The current collector arrangement includes a first current collector element forming a first current collection section having a first free end and a second current collector element forming a second current collection section having a second free end, wherein the first and second current collection sections each extend in or on the electrode body such that the first free end of the first current collector element and the second free end of the second current collector element are arranged at a distance with respect to one another in or on the electrode body.

### CROSS-REFERENCE TO RELATED APPLICATIONS

This application is the United States National Phase under 35 U.S.C. § 371 of PCT International Patent Application No. PCT/EP2023/073565, filed on Aug. 28, 2023, which claims the benefit of European Patent Application No. 22198947.8, filed on Sep. 30, 2022, the disclosures of which are hereby incorporated by reference herein in their entireties.

### TECHNICAL FIELD

The instant invention concerns an electrical energy storage device for an implantable medical stimulation device according to the preamble of claim 1.

### BACKGROUND

An electrical energy storage device of this kind may for example be formed by an electrochemical battery or an electric capacitor, in particular an electrolytic capacitor.

An electrical energy storage device of the type concerned herein generally comprises a housing, a first electrical electrode and a second electrical electrode having different electrical polarities, one of the first electrical electrode and the second electrical electrode being formed by an electrode element having an electrically conductive electrode body arranged in the housing. A current collector arrangement is electrically connected to the electrode element for collecting and/or conducting electrical charges, e.g. electrons, at the electrode element in order to provide electrical energy from the electrical energy storage device.

An electrode body of an electrode element as concerned herein may formed as a massive or solid body forming a block-like element, which serves as an electrode active material during operation of the electrical energy storage device. The electrode element may for example be formed by employing a sintering technique, in the course of which particles are compressed and partially melted in order to increase the density of the material and to form bridges between particles. By sintering, a compact metal part is formed from an originally porous powder material. Such sintered electrode bodies are particularly used when tantalum or niobium are used as electrode active materials.

Alternatively, the electrode body may be designed in form of a foil or a sheet made from a suitable electrode active material, e.g. a metal such as an alkaline metal (lithium, sodium) or aluminum.

Also, as an alternative, the electrode body may be formed from an electrode active material being in a powder or granular form, wherein the electrode active material is pressed into the desired form, however, without being sintered afterwards. Such manufacturing is commonly used for electrodes comprising carbon (graphite, carbon monofluoride or the like), manganese dioxide or other mixed metal oxides as electrode active material.

Typically, within a current collector arrangement an electrode element is used to collect electrical charges, e.g. electrons, at the electrode element, the electrode element having the shape of a wire or a mesh for example embedded within the material of the electrode element or arranged on or at the electrode element. Dependent on the electrical characteristics of the electrode body, this may cause collection of electrical charges in particular in the vicinity of the current collector element and may have the effect that efficiency of electrical charge collection is not optimal, such that the stored energy of the electrical energy storage device may not be optimally exploited, for example when using the electrical energy storage device for a high-current discharge to generate a stimulation shock in an implantable medical stimulation device, such as an implantable defibrillator device.

US 2018/0159113 A1 describes an electrical energy storage device having the shape of a battery comprising an electrode assembly housed inside a casing.

### SUMMARY

It is an object of the instant invention to provide an electrical energy storage device, an assembly of at least two electrical energy storage devices and an implantable medical stimulation device comprising an electrical energy storage device or an assembly of at least two electrical energy storage devices which allow, in an easy and efficient way, to exploit stored energy by efficiently collecting and/or conducting electrical charges, e.g. electrons, on an electrode element.

This object is achieved by means of an electrical energy storage device comprising the features of claim 1.

Accordingly, the current collector arrangement comprises a first current collector element forming a first current collection section having a first free end and a second current collector element forming a second current collection section having a second free end. The first current collection section and the second current collection section each extend in the electrode body or along a surface of the electrode body such that the first free end of the first collector element and the second free end of the second connector element are arranged at a distance with respect to one another in or on the electrode body.

The electrical energy storage device comprises a first electrode and a second electrode having different polarities, one of which is formed by an electrode element having an electrode body, in particular a massive or solid electrode body, for example formed by a sintering technique. The electrode body, as a whole, is electrically conductive such that, by means of the electrode body, electrical charges, e.g. electrons may be collected and/or conducted in order to harvest energy from the electrical energy storage device.

Herein, in order to collect and/or conduct electrical charges and harvest electrical energy at the electrode body, a current collector arrangement is used, which comprises (at least) a first current collector element forming a first current collection section and a second current collector element forming a second current collection section. Hence, at least two current collector elements are used on the (single) electrode element, the current collection sections being arranged in the electrode body or on a surface of the electrode body such that the current collection sections effectively are coupled to the electrode body.

As (at least) two current collector elements are used on the electrode element for collecting or conducting electrical charges during operation of the electrical energy storage device, efficiency of collecting and/or conducting electrical charges and hence the efficiency of harvesting and exploiting stored energy from the electrical energy storage device may be improved. In particular, as electrical charges are predominantly collected and/or conducted by the current collector elements in the vicinity of the current collection sections and hence in a region surrounding the collection sections, the effective area for collecting electrical charges may be increased by increasing the number of current collector elements.

It is to be noted that the current collector arrangement may comprise two or more current collector elements. In particular it also is conceivable to use more than two current collector elements, for example three, four or five current collector elements for establishing a connection to the (single or individual) electrode element.

The electrical energy storage device in particular may be used in an implantable medical stimulation device, for example an implantable medical stimulation device providing for a cardiac stimulation function, such as an implantable defibrillator or the like.

The first current collector element and the second collector element, with their current collection sections, are arranged in or on the electrode body of the electrode element, such that their free ends are spatially separate from one another. The current collection sections of the current collector elements hence, except for the connection provided by the electrode body, are structurally separate from one another such that the first free end of the first current collection section of the first current collector element and the second free end of the second current collection section of the second current collector element are not structurally connected, by a structure of the current collector arrangement itself, within the electrode body, except by the electrode body.

The current collector elements hence form separate current collection sections extending in and across different regions of the electrode body, such that electrical charges may be effectively collected and/or conducted at the electrode body in a wide area across the electrode body.

In one embodiment, the first current collection section and the second current collection section are not structurally connected to each other within the volume of the electrode body, except by the electrode body. Other than via the electrode body, hence, there is no structural connection in between the first current collection section and the second current collection section within the volume of the electrode body. The current collector elements, with the free ends of the current collection sections, hence, are free from one another.

In one embodiment, the electrode body comprises a first (effective) electrical conductivity, and the first current collector element and the second current collector element each comprise a second (effective) electrical conductivity, wherein the first (effective) electrical conductivity is smaller than the second (effective) electrical conductivity. The current collector elements, in comparison to the electrode body, thus provide for a reduced electrical resistance and hence allow for an electrical charge or current conduction in the electrode body and away from the electrode body with a reduced electrical resistance as compared to a conduction in the material of the electrode body. The current collector elements hence are operative to collect and/or conduct electrical charges within the electrode body, such that energy may efficiently be harvested by providing an electrical current from the energy storage device. As multiple current collector elements having multiple current collection sections extending in or on the electrode body are provided, multiple current paths formed by the current collector elements are provided within or on the electrode body for collecting and/or conducting electrical charges.

The electrode body may for example be formed by employing a sintering technique, in which particles, for example a particle powder, is compressed and heated such that particles are partially melted and bonded to each other, and a dense, compact body is formed. Such sintering technique is particularly appropriate in this case, the particle powder essentially consists of or comprises tantalum or niobium.

Alternative, the electrode body may be formed by pressing a composition comprising an electrode active particle powder and optionally a binder and/or conductive additive, e.g. made from carbon (e.g. graphite, graphene or hardened carbon) into a desired form, particularly in case of the electrode active material essentially consists of or comprises graphite, carbon monofluoride or manganese dioxide.

As a further alternative, the electrode body may be formed by a foil or sheet of electrode active material, wherein the electrode active material may comprise aluminum, or an alkaline metal, particularly lithium, sodium, or potassium.

In one embodiment, the first current collection section of the first current collector element and the second current collection section of the second current collector element are each embedded within the material of the electrode body. When manufacturing the electrode body, hence, the current collection sections of the current collector elements are placed within the material of the electrode body, such that the current collection sections are integrally embedded within the electrode body, for example when sintering the electrode body.

In one embodiment, the first current collection section and the second current collection section extend along different directions in or on the electrode body. For example, the current collector elements may reach into the electrode body along opposite directions, such that the first current collector element is arranged at a first face of the electrode body and the second current collector element is arranged at a second, opposite face of the electrode body. In another embodiment, the current collector elements may be arranged to extend along different directions arranged at an angle with respect to one another in or on the electrode body.

The electrical energy storage device comprises a first electrical electrode and a second electrical electrode which are at different polarities (+vs. −) . One of the electrical electrodes forms a cathode, whereas the other electrical electrode forms an anode. For example, the electrode element may function as anode within the electrical energy storage device, such that the other electrode element serves as cathode.

In one embodiment, the housing is made from an electrically conductive material, wherein the other of the first electrical electrode and the second electrical electrode is formed by the housing or is electrically connected to the housing. The housing encloses the electrode element, but is electrically separate from the electrode element, the housing for example forming or being connected to the cathode of the electrical energy storage device. Not limiting examples for suitable electrically conductive materials include titanium, a titanium alloy, aluminum, or stainless steel. For example, in one embodiment, an electrical connection is established between the housing and an associated electrical electrode inside the housing, such that via the housing the electrical electrode associated with the housing may be electrically contacted.

In one embodiment, the current collector arrangement comprises at least one output section. The housing comprises at least one feedthrough for guiding the at least one output section through the housing, the at least one feedthrough providing for an electrical insulation of the at least one output section with respect to the housing such that an effective electrical coupling to the electrode element received within the housing may be established using the at least one output section.

An electrical connection to the electrical electrodes of the electrical energy storage device may for example be established by using connection lines, such as connective bands, wires or strips, in particular flexible strips, or printed circuit boards, or insulated conductors. Connection lines may for example be connected to the housing for establishing an electrical connection to the electrical electrode associated with the housing, and to one or multiple output sections of the collector elements associated with the electrode element received within the housing for establishing an electrical connection to the electrode element within the housing.

In one embodiment, the housing is hermetically tight, i.e. fluid tight. For this, in particular also the at least one feedthrough is hermetically tight such that the output section is guided through the housing via the feedthrough while not impacting the hermetic tightness of the housing.

The feedthrough is for example made from or comprises a ceramic, a glass or glass solder, or a polymer, particularly an epoxy resin, silicone, or a liquid crystal polymer.

Within the meaning of the present invention, the term “liquid crystal polymer” is used in the meaning known to and commonly used by a person skilled in the art. A “liquid crystal polymer” refers in particular to an aromatic polymer, which has highly ordered or crystalline regions in the molten state or in solution. Non-limiting examples include aromatic polyamides such as aramid (Kevlar) and aromatic polyesters of hydroxybenzoic acid, such as a polycondensate of 4-hydroxybenzoic acid and 6-hydroxynaphthalene-2-carboxylic acid (Vectran).

The feedthrough is inserted into an opening formed in the housing such that, beneficially, the feedthrough provides for an electrical insulation of the output section with respect to the housing and at the same time tightly closes the opening in the housing.

The current collector arrangement may employ, in one embodiment, a single output section, which within the housing is electrically coupled to both current collector elements. In this embodiment, a single feedthrough for guiding one output section through the housing is sufficient.

In another embodiment, the current collector arrangement may employ multiple output sections, for example one output section associated with each current collector element. In this embodiment no electrical coupling in between the current collector elements within the housing is required, but rather output sections are separately coupled each to an associated current collector element and are separately guided, by using a feedthrough for each, through the housing.

In one embodiment, the electrical energy storage device is designed as an electrochemical cell, particularly as a battery. In another embodiment, the electrical energy storage device is designed as a capacitor, particularly as an electrolytic capacitor.

In one embodiment in which the electrical energy storage device is designed in as a battery, one of the electrical electrodes comprises an alkali metal, particularly selected from lithium, sodium or potassium. The battery may be a primary battery or a secondary battery.

In one embodiment, the electrical energy storage device is designed as primary battery, wherein one of the electrical electrodes comprises for example an alkali metal as an electrode active material, particularly selected from lithium, sodium or potassium, and the other of the electrical electrodes comprises as an electrode active material carbon monofluoride (CFx), manganese dioxide (MnO₂), graphite, iodine (I₂), silver vanadium oxide (SVO), copper silver vanadium oxide (CSVO), V₂O₂, TiS₂, CuO₂, CuzS, FeS, FeS₂, Ag₂O, Ag₂O₂, CuF, Ag₂CrO₄, CuO, CuzP₂O₇, Cu₄P₂O₉, CusP₂O₁₀, Ag₂Cu₂P₂O₈, Ag₂Cu₃P₂O₉, copper vanadium oxide or a mixture thereof.

In one embodiment, the electrical energy storage device is designed as a secondary battery, wherein one of the electrical electrodes for example comprises an alkali metal as an electrode active material, particularly selected from lithium, sodium, potassium or carbon, particularly graphite, hard carbon or non-graphitizing carbon, and the other of the electrical electrodes comprises as an electrode active material particularly lithium cobalt oxide (LiCoO₂), lithium nickel manganese cobalt oxide (mixed oxides of LiCoO₂, LiNiO₂ and LiMnO₂), lithium nickel cobalt aluminum oxide (NCA), lithium manganese oxide (LiMn₂O₄), or lithium iron phosphate (LiFePO₄).

In one embodiment, the electrical energy storage device is designed as a battery (primary battery or secondary battery) and further comprises an electrolyte, preferably a non-aqueous electrolyte, wherein particularly one of the electrical electrodes comprises as an electrode active material an alkali metal, particularly selected from lithium, sodium or potassium. Suitable electrolytes include, without being restricted to, non-aqueous, preferable aprotic, solvents, such as an ester, an ether and a dialkyl carbonate, particularly tetrahydrofuran, methyl acetate, diglyme (bis(2-methoxyethyl)ether), triglyme (tris(2-methoxyethyl)ether), tetraglyme (tetra(2-methoxyethyl)ether), 1,2-dimethoxyethane, 1,2-diethoxyethane, 1-ethoxy-2-methoxyethane, dimethyl carbonate, diethyl carbonate, dipropyl carbonate, ethyl methyl carbonate, methyl propyl carbonate, ethyl propyl carbonate or a mixture thereof, or a cyclic carbonate, a cyclic ester, a cyclic amide, particularly propylene carbonate, ethylene carbonate, butylene carbonate, y-butyrolactone, N-methyl pyrrolidinone or a mixture thereof. Suitable electrolytes also comprise polar non-aqueous solvents such as acetonitrile, dimethyl sulfoxide, dimethyl formamide, dimethyl acetamide or a mixture thereof.

In one embodiment, the electrolyte comprises at least one conductive salt. The conductive salt is preferably an inorganic alkali metal salt, whereby the alkali metal cation is the same as the active anode material. Suitable anions are PF₆⁻, BF₄⁻, AsF₆⁻, SbF₆⁻, ClO₄⁻, O₂⁻, AlCl₄⁻, GaCl₄⁻, SCN⁻, SO₃(C₆F₅)⁻, C(SO₂CF₃)₃⁻, N(SO₂CF₃)₂⁻ and SO₃CF₃ among others. In a preferred embodiment, the concentration of the conductive salt is, LiPF₆, LiClO₄, LiFSI, LiTFSI, LiAsF₆, LiBOB, LiODFB, or LiNO₃.

In one embodiment, the electrical energy storage device is designed as an electrolytic capacitor, wherein one of the electrical electrodes comprises as an electrode active material for example tantalum or niobium, and the other of the electrical electrodes comprise as an electrode active material for example activated carbon, graphite, graphene, a carbon nanotube, and a conductive polymer.

In an embodiment in which the electrical energy storage device is designed as an electrolytic capacitor, the housing is electrically conductive and e.g. is made from aluminum, titanium, a titanium alloy, or stainless steel, wherein one of the electrical electrodes of the electrical energy storage device is formed by a layer or coating applied on an inner surface of the conductive housing, the layer or coating being in electrically conductive communication with the conductive housing. The layer of coating may be attached to the inner surface of the conductive housing by means of an adhesion layer. The layer or coating may further comprise a binder such as polyvinylidene fluoride (PVDF) polytetrafluoroethylene (PTFE), carboxymethyl cellulose (CMC) or a rubber, particularly acryl rubber, nitrile butadiene rubber, styrene butadiene rubber (SBR) or butyl rubber.

In one embodiment in which the electrical energy storage device is designed as an electrolytic capacitor, one of the electrical electrodes comprises, as an electrode active material, aluminum, preferably an aluminum foil, and the other of the electrical electrodes comprises, as an electrode material, aluminum, preferably an aluminum foil, wherein particularly a separator is arranged between the electrical electrodes. The separator may e.g. be made from an absorbent paper.

In one embodiment in which the electrical energy storage device is designed as an electrolytic capacitor, the electrical energy storage device comprises an aqueous electrolyte. In one embodiment, the electrolyte comprises ethylene glycol and optionally an acid, particularly boric acid or acetic acid. One of the electrical electrodes, for example the electrode element received in the housing, may e.g. comprise aluminium or tantalum as electrode active material. In one embodiment, the electrolyte comprises ethylene glycol, acetic acid and ammonium acetate. In one embodiment, the electrolyte comprises dimethylformamide, dimethylacetamide and/or γ-butyrolactone. In one embodiment, the electrolyte comprises tetracyanoquinodimethane, polypyrrole, or poly(3,4-ethylenedioxythiophene).

In one embodiment, the housing comprises at least one rounded edge. In particular, the housing may comprise a rounded face. In one embodiment, the housing comprises a planar face at one side and the rounded face at an opposite side. The rounded face may in particular be adapted such that it conforms with the shape of a generator device housing of an implantable medical stimulation device, such that the energy storage device may be tightly fitted in a cavity of the generator device housing.

The rounded edge of the housing may in particular comprise a radius of curvature of at least 0.5 mm, preferably larger than 1 mm, even more preferably larger than 2.5 mm.

In one embodiment, the housing, the electrode element and the current collector arrangement comprising the first current collector element and the second current collector element are arranged symmetrically with respect to a plane of symmetry, the first current collector element being arranged at a first side of the plane of symmetry and the second current collector element being arranged at a second side of the plane of symmetry. In particular, the entire electrical energy storage device may exhibit a symmetry with respect to the plane of symmetry, wherein the symmetry is such that the collector elements are arranged symmetrically, with respect to the plane of symmetry, on the electrode body of the electrode element and, beneficially, output sections are symmetrically guided through the housing of the electrical energy storage device by means of corresponding feedthroughs at symmetrical locations at a first side of the plane of symmetry and at a second side of the plane of symmetry.

By forming the electrical energy storage device symmetrically with respect to a central plane of symmetry, it in particular may be facilitated to combine multiple energy storage devices to form an assembly of energy storage devices to increase the overall energy storage capacity. Due to the symmetric structure of the energy storage devices, output sections for establishing an electrical connection in between the different energy storage devices are symmetrically placed on the housing of the energy storage devices, such that an electrical connection in between the electrical energy storage devices may easily and space-efficiently be established.

In one embodiment, the electrical energy storage device comprises a connection line which electrically connects the first current collector element and the second current collector element. The connection line is arranged outside of the electrode body and hence is separate from the electrode body. The connection line may be for example received within the housing and hence may run inside the housing. In another embodiment, the connection line may extend outside of the housing and may provide for an electrical connection in between the collector elements outside of the housing.

In one embodiment, the connection line is placed on a carrier element attached to the housing, for example outside of the housing. The carrier element may for example be formed by a printed circuit board (PCB), wherein by means of the connection line an electrical connection in between the different collector elements, for example between output sections associated with the different collector elements, is established.

In one embodiment, an assembly of at least two electrical energy storage devices is used to provide for an increased energy storage capacity. The electrical energy storage devices may for example be electrically connected by a connection arrangement, which employs a multiplicity of connection lines which establish a functional, electrical connection in between the energy storage devices.

In particular, by means of the connection arrangement the at least two electrical energy storage devices may electrically be connected in series, such that the output voltages of the electrical energy storage devices add to one another. Alternatively, by means of the connection arrangement the at least two electrical energy storage devices may electrically be connected in parallel.

In one embodiment, the housings of a first of the at least two electrical energy storage devices and a second of the at least two electrical energy storage devices each comprise a rounded face and a planar face. The first and the second of the at least two energy storage devices are arranged with respect to one another such that the housings of the first and the second of the at least two energy storage devices face towards each other with the planar faces and face away from one another with the rounded faces. In this way it becomes possible to form an assembly of electrical energy storage devices which may be placed on one another such that the planar faces of the energy storage devices abut one another. In that the rounded faces point outwards, the outer shape of the assembly may be such that it conforms to the shape of a generator device housing and hence may be efficiently received in the generator device housing, hence optimally using a space available within the generator device housing.

In one embodiment, an implantable medical stimulation device comprises an electrical energy storage device according to the type described above or an assembly of at least two electrical energy storage devices according to the type described above.

### BRIEF DESCRIPTION OF THE DRAWINGS

The idea of the invention shall subsequently be described in more detail with reference to the embodiments shown in the figures. Herein:

FIG. 1 shows a schematic view of an implantable medical stimulation device having a generator device;

FIG. 2 shows a schematic drawing of an electrical energy storage device for use in an implantable medical stimulation device;

FIG. 3 shows a schematic view of another embodiment of an electrical energy storage device;

FIG. 4 shows an embodiment of an assembly of electrical energy storage devices;

FIG. 5 shows the assembly of FIG. 4, in an electrically connected state;

FIG. 6 shows another example of an assembly of electrical energy storage devices;

FIG. 7 shows yet another example of an assembly of electrical energy storage devices;

FIG. 8 shows yet another example of an assembly of electrical energy storage devices;

FIG. 9 shows another embodiment of an electrical energy storage device;

FIG. 10 shows yet another embodiment of an electrical energy storage device;

FIG. 11 shows yet another embodiment of an electrical energy storage device;

FIG. 12 shows a schematic drawing of an electrode element with a collector element arranged thereon;

FIG. 13A shows an embodiment of an electrode element with a collector element arranged thereon;

FIG. 13B shows another embodiment of a collector element arranged on an electrode element; and

FIG. 13C shows yet another embodiment of a collector element arranged on an electrode element.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

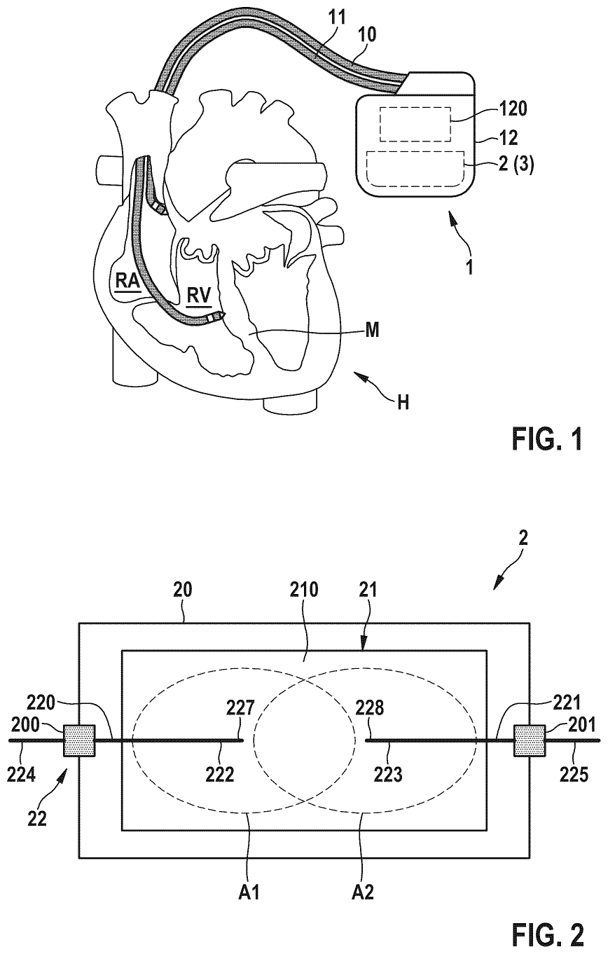

FIG. 1 shows, in a schematic drawing, an embodiment of an implantable medical stimulation device 1 implanted in a patient, the implantable medical stimulation device 1 comprising a generator 12 connected to leads 10, 11 extending from the generator 12 through the superior vena V into the patient's heart H. By means of the leads 10, 11, electrical pulses for providing a pacing action and/or defibrillation in the heart H may be delivered to intra-cardiac tissue potentially at different locations within the heart, and sense signals may be received.

In the embodiment of FIG. 1, an electrode lead 10 is implanted into the heart H such that it extends into the right ventricle RV of the heart H and is arranged on intra-cardiac tissue M in the right ventricle RV of the heart H. An electrode lead 11 in turn is implanted such that it reaches into the right atrium RA.

An implantable medical stimulation device 1 as concerned herein may generally be a cardiac stimulation device, such as a cardiac pacemaker or defibrillator device, e.g. a one-chamber or two-chamber implantable pulse generator (IPG), or a one-chamber or two-chamber implantable cardioverter defibrillator (ICD). A stimulation device 1 of this kind may comprise a generator 12, as shown in FIG. 1, which may be subcutaneously implanted in a patient at a location remote from the heart H, and one or multiple leads 10, 11 extending from the generator 12 into the heart H for emitting stimulation pulses in the heart H or for obtaining sense signals at one or multiple locations from the heart H. The leads 10, 11 each form a generally longitudinal, tubular body, which reaches into the heart H and is anchored at a location of interest within the heart H.

As is schematically illustrated in FIG. 1, the generator device 12 comprises processing circuitry 120 which is configured to control operation of the implantable medical stimulation device 1, in particular for processing sensed signals and for generating stimulation signals for performing a stimulation action on the patient's heart H. In addition, the generator device 12 comprises an electrical energy storage device 2 or an assembly 3 of electrical energy storage devices 2 which serve to supply electrical energy for enabling operation of the implantable medical stimulation device 1.

For example, in case the implantable medical stimulation device 1 is configured to perform a defibrillation function, the energy storage device 2 or the assembly 3 of energy storage devices 2 is configured to generate one or multiple shock pulses in order to output defibrillation shocks.

As the available space within a generator device housing inherently is limited and thus also the space available for the electrical energy storage device 2 or the assembly 3 of electrical energy storage devices 2 within the generator device housing is limited, there is a general desire to provide an electrical energy storage device 2 or an assembly 3 of electrical energy storage devices 2 which are enabled to provide for a sufficient energy storage capacity, at a large energy density and the possibility for an efficient energy exploitation for exploiting stored energy from the electrical energy storage device 2 or the assembly 3 of electrical energy storage devices 2.

Referring now to FIG. 2, in one embodiment an electrical energy storage device 2 comprises a housing 20 which receives an electrode element 21 therein.

The housing 20 may serve or may be electrically connected to a first electrical electrode, the electrode element 21 serving as another, second electrical electrode having a different polarity in comparison to the electrical electrode formed by or connected to the housing 20.

The electrode element 21 comprises an electrode body 210 which may be formed, as schematically indicated in FIG. 12, by a massive block element which for example is formed by a sintering technique from an electrically conductive material, such as an niobium or tantalum material. Alternatively, the electrode body 120 may be designed in form of a foil or sheet made from, e.g. aluminum.

In order to collect electrical charges, e.g. electrons, on the electrode element 21 for exploiting energy from the energy storage device 2, a current collector arrangement 22 comprises current collector elements 220, 221 which are arranged on the electrode body 210 of the electrode element 21, such that electrical charges may be received at the current collector elements 220, 221 and a current may be output by the electrical energy storage device 2 via the collector elements 220, 221. Whereas in conventional electrical energy storage devices 2 as for example found in an implantable medical stimulation device 1 only a single current collector element is used on an (single, individual) electrode element 21 received within a housing 20, it herein is proposed to use (at least) two current collector elements 220, 221 to harvest energy from the electrical energy storage device 2. The current collector elements 220, 221 each comprise a current collection section 222, 223 which reaches into the electrode body 210 of the electrode element 21, the current collection sections 222, 223 in each case forming a free end 227, 228 which is placed within the electrode body 210 or on a surface of the electrode body 210.

In particular, the current collection sections 222, 223 may be embedded within the material of the electrode body 210, as this is schematically illustrated in FIG. 12. For example, the current collection sections 222, 223 may be integrally received and embedded within the electrode body 210 in that the current collection sections 222, 223 of the current collector elements 220, 221 are placed within the material of the electrode body 210 during manufacturing of the electrode body 210 and are integrally connected to the electrode body 210 for example when sintering the electrode body 210.

By means of the current collector elements 220, 221, typically electrical charges are collected in a spatial region A1, A2 in the vicinity of the current collection sections 222, 223. In that multiple current collector elements 220, 221 are employed, the effective region increases from which electrical charges may be collected on the electrode body 210 of the electrode element 21, such that the energy exploitation of the electrical energy storage device 2 may be increased in that stored energy may be efficiently harvested from the electrical energy storage device 2, for example for use in an implantable medical stimulation device 1 such as an implantable defibrillator.

It is to be noted that in the embodiment of FIG. 2 and also in other embodiments described herein two current collector elements 220, 221 are used on a (single, individual) electrode element 21. However, also more than two collector elements 220, 221 may be employed, for example three or more current collector elements.

In the embodiment of FIG. 2, the housing 20 is made from an electrically conductive material and forms or is connected to an electrical electrode serving as a counter electrode to the electrical electrode formed by the electrode element 21. Output sections 224, 225 of the collector elements 220, 221 are guided through the housing 20 at feedthroughs 200, 201, the feedthroughs 200, 201 providing for an electrical insulation of the output sections 224, 225 with respect to the housing 20.

The feedthroughs 200, 201 may for example be made from a glass or ceramic material, or a polymer material.

In one embodiment, the housing 20 is hermetically tight, in particular fluid tight, such that liquid and gas may not enter into our exit from the housing 20. The tight sealing of the housing 20 herein is not impacted by the feedthroughs 200, 201, which provide for a fluid-tight guidance of the output sections 224, 225 through the housing 20.

In the embodiment of FIG. 2, the current collector elements 220, 221 of the current collector arrangement 22 are arranged such that they are aligned with their current collection sections 222, 223, the free ends 227, 228 being arranged at a distance within the electrode body 210 of the electrode element 21. The current collection sections 222, 223 herein extend along opposite directions into the electrode body 210.

The current collector elements 220, 221, at their current collection sections 222, 223 within the electrode body 210, are structurally not connected to each other, except by the electrode body 210. The free ends 227, 228 hence are structurally separate from one another in that no structure of the current collector arrangement 22 provides for an interconnection in between the collection sections 222, 223 within the electrode body 210.

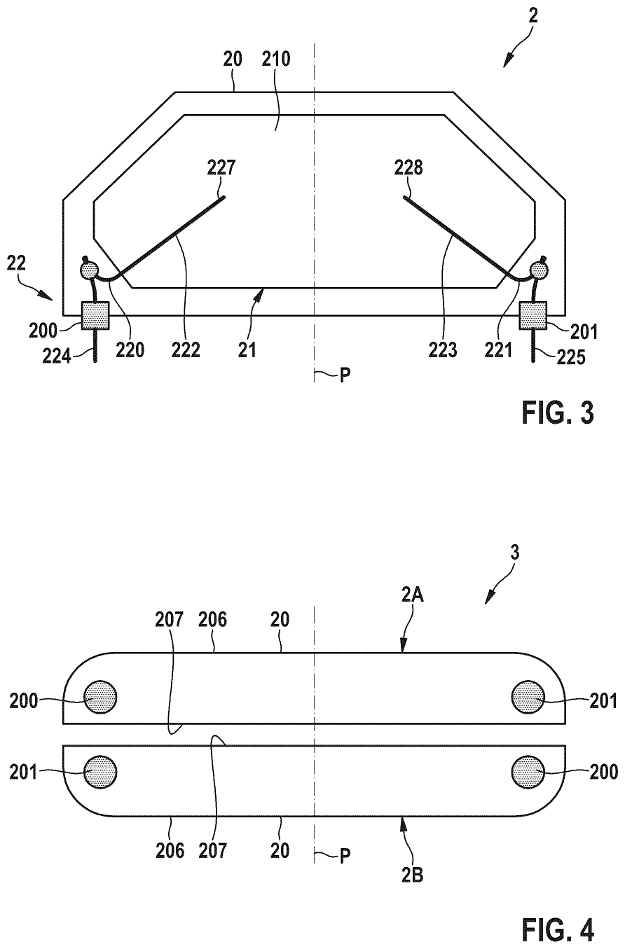

Referring now to FIG. 3, in another embodiment the housing 20 and the electrode element 21 may differ from a rectangular (cubic) shape. Rather, the housing 20 and the electrode element 21 in a top view may have a polygonal shape, hence allowing to adapt the shape of the electrical energy storage device 2 to the shape of a cavity within a generator device housing of a generator device 12, such that the electrical energy storage device 2 may be space-efficiently received within the generator device housing.

In the embodiment of FIG. 3, current collection sections 222, 223 of the current collector elements 220, 221 are arranged at an angle with respect to one another, wherein like in the embodiment of FIG. 2 the current collection section 222, 223 are structurally separate from one another except for the connection provided by the embedding within the material of the electrode body 210.

In the embodiment of FIG. 3, output sections 224, 225 are guided through the housing 20 at feedthroughs 200, 201 at a common face of the housing 20, such that the output sections 224, 225 in parallel extend from the housing 20 and are accessible at the common face.

In the embodiment of FIG. 3, the electrical energy storage device 2 is formed symmetrically with respect to a plane of symmetry P. The plane of symmetry P is oriented perpendicularly in between the current collector elements 220, 221, such that the current collector elements 220, 221 are arranged at opposite sides with respect to the plane of symmetry P. The output sections 224, 225 on the feedthroughs 200, 201 are arranged at either side of the plane of symmetry P and are equidistantly placed with respect to the plane of symmetry P. In addition, the housing 20 and the electrode element 21 are symmetrical with respect to the plane of symmetry P.

Referring now to FIG. 4, a symmetry of the electrical energy storage device 2 allows to form an assembly 3 of multiple electrical energy storage devices 2A, 2B, which may be received in a space-efficient manner in a generator device housing of a generator device 12 of an implantable medical stimulation device 1.

As shown in FIG. 4, the housings 20 of the electrical energy storage devices 2A, 2B may each comprise a planar face 207. The planar faces 207 of the housings 20 may be abutted on one another for forming the assembly 3, such that the electrical energy storage devices 2A, 2B may be arranged on one another in a space-efficient manner.

In addition, each housing 20 comprises, facing away from the planar face 207, a rounded face 206 which faces outwards. An outer envelope of the assembly 3 herein is formed by the rounded faces 206 of the housings 20 of the individual electrical energy storage devices 2A, 2B, the shaping of the rounded faces 206 allowing for adapting the assembly 3 to the shape of a cavity within a generator device housing to space-efficiently receive the assembly 3 within the generator device housing.

As visible from FIG. 4, due to the symmetric arrangement of the feedthroughs 200, 201 and the output sections 224, 225 on the housings 20 of the electrical energy storage devices 2A, 2B, the feedthroughs 200, 201 and thus the output sections 224, 225 of the electrical energy storage devices 2A, 2B are aligned with one another and are arranged proximally with respect to one another when forming the assembly 3. This allows to establish an electrical connection in between the electrical energy storage devices 2A, 2B by employing short electrical connection lines, thus using minimum space for establishing an electrical connection.

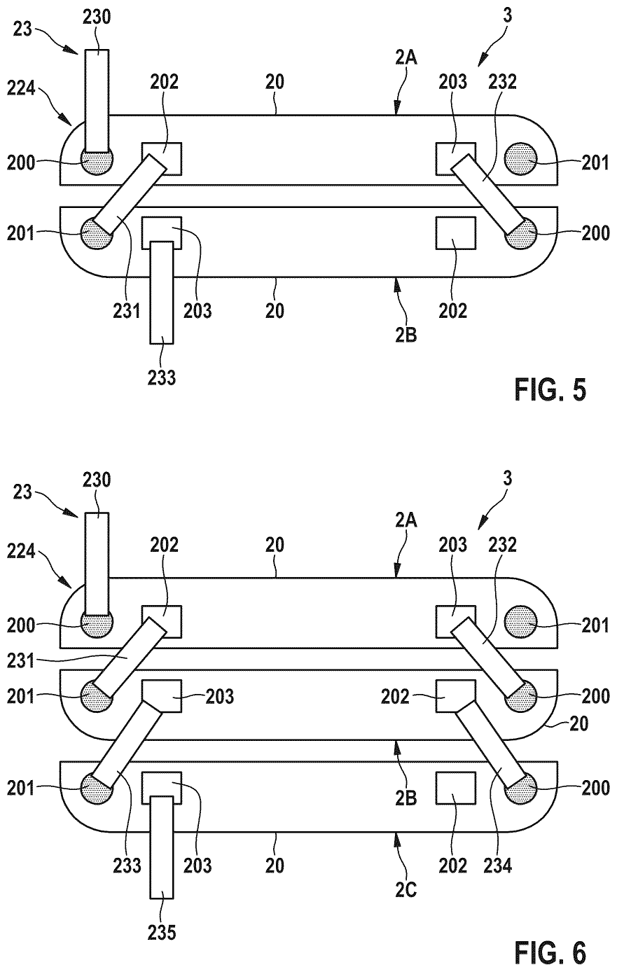

Referring now to FIG. 5, in one embodiment the electrical energy storage devices 2A, 2B of the assembly 3 are electrically connected in series. For this, a connection arrangement 23 is formed by a set of connection lines 230-233 such that output voltages of the electrical energy storage devices 2A, 2B add up according to the series connection.

In the embodiment of FIG. 5, connection terminals 202, 203 are formed on each housing 20 of the electrical energy storage devices 2A, 2B, the terminals 202, 203 of the housing 20 of the electrical energy storage devices 2A being connected to output sections 224, 225 at the feedthroughs 200, 201 of the housing of the electrical energy storage device 2B. Output terminals of the assembly 3 are provided by connection lines 230, 233, the connection line 230 being connected to the output section 224 at the feedthrough 200 of the electrical energy storage device 2A and the connection line 233 being connected to the connection terminal 203 on the housing 20 of the electrical energy storage device 2B. The connection lines 230, 233 hence provide for output terminals at opposite polarities at which the combined output voltage of both electrical energy storage devices 2A, 2B is output.

The connection lines 230-233 may for example be formed by connective bands or strips, such as flexible strips, or by printed circuit boards. In another embodiment, electrically insulated lines, for example guided in a plastic or ceramic insulation, may be used.

Whereas in the embodiment of FIG. 5 two electrical energy storage devices 2A, 2B are combined to form the assembly 3, in the embodiments of FIGS. 6 to 8 three electrical energy storage devices 2A, 2B, 2C are combined to form a respective assembly 3.

In the embodiment of FIG. 6, an additional, third electrical energy storage device 2C is added to the assembly of FIG. 5, in that the electrical energy storage device 2C by connection lines 233, 234 is connected to the electrical energy storage device 2B, namely by connecting the housing 20 of the electrical energy storage device 2B to the output sections 224, 225 at the feedthroughs 200, 201 of the electrical energy storage device 2C. By means of a connection line 235 connected to the connection terminal 203 on the housing 20 of the electrical energy storage device 2C an output terminal for the assembly 3 is formed on the electrical energy storage device 2C, such that in between the output terminals formed by the connection lines 230, 235 the combined output voltage of the three electrical energy storage devices 2A, 2B, 2C is output.

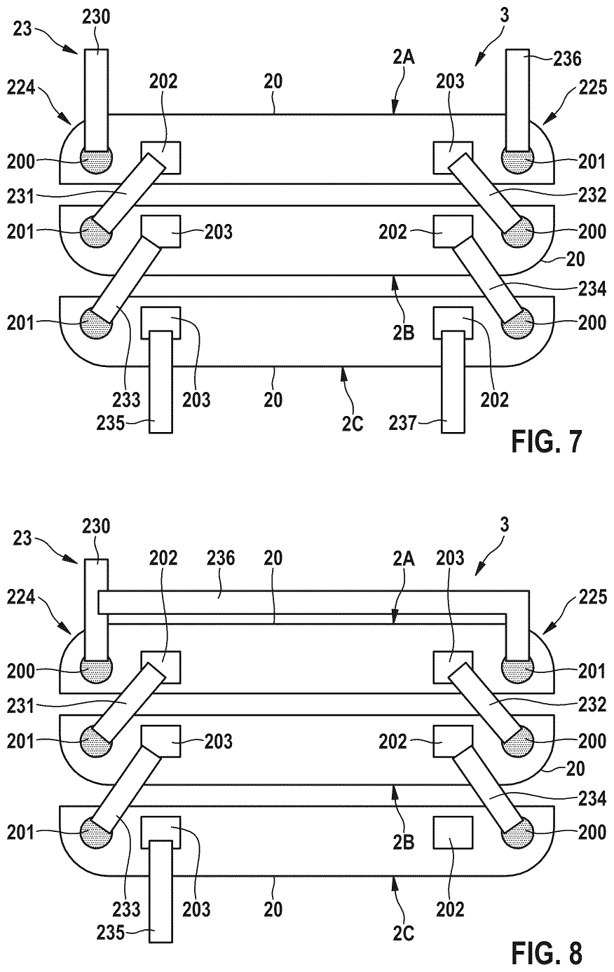

The embodiment of FIG. 7 differs from the embodiment of FIG. 6 in that additional connection lines 236, 237 are used to provide for additional output terminals of the assembly 3. Connection line 236 is connected to the output section 225 at the feedthrough 201 of the electrical energy storage device 2A, whereas connection line 237 is connected to a connection terminal 202 on the housing 20 of the electrical energy storage device 2C.

Hence, at the electrical energy storage device 2A two first output terminals corresponding to the polarity of the output sections 224, 225 on the feedthroughs 200, 201 of the electrical energy storage device 2A are provided in parallel. Likewise, on the electrical energy storage device 2C two second output terminals corresponding to the polarity of the housing 20 of the electrical energy storage device 2C are provided in parallel.

The embodiment of FIG. 8 differs from the embodiment of FIG. 6 in that an additional connection line 236 is provided which connects the output section 225 at the feedthrough 201 to the output section 224 at the feedthrough 200 on the electrical energy storage device 2A, such that the electrode elements 220, 221 associated with the output sections 224, 225 are electrically connected to one another outside of the electrical energy storage device 2A.

In general, the different collector elements 220, 221 of an electrical energy storage device 2 may be electrically connected to one another within the housing 20 of the electrical energy storage device 2 or outside of the housing 20.

In the shown embodiments, connection terminals 202, 203 on the housing 20 of a particular electrical energy storage device 2A, 2B, 2C may be configured to establish for example a soldering connection to the associated connection lines 230-237. Alternatively, plug-in connections may be established by using suitable electrical connectors.

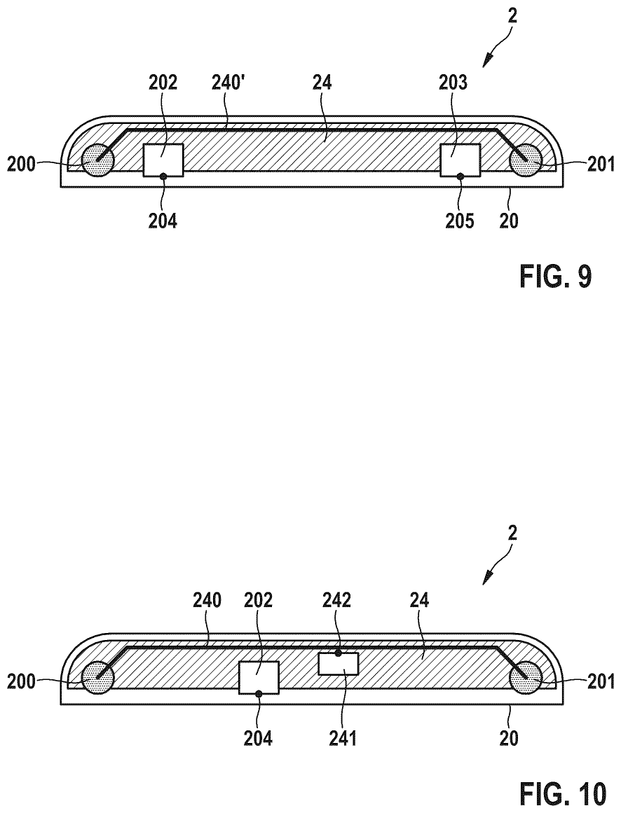

Likewise, soldering connections or plug-in connections may be established in between the associated connection lines 230-237 and the output sections 224, 225 at the feedthroughs 200, 201. Referring now to FIG. 9, a connection of current collector elements 220, 221 may for example be established outside of the housing 20 by using a connection line 240 running in between the feedthroughs 200, 201 and the corresponding output sections 224, 225 of the current collector elements 220, 221 arranged thereon. In one embodiment, the connection line 240 is arranged on a carrier element 24, for example a printed circuit board, which is placed on and connected to the housing 20.

In addition, connection terminals 202, 203 for establishing an electrical connection to the housing 20 may be provided on the carrier element 24, the connection terminals 202, 203 being electrically connected to the housing 20 for example by electrical interconnections, for example soldering connections.

In the shown embodiment, the connection terminals 202, 203 are arranged in the vicinity to the feedthrough 200, 201, such that two electrical interconnections to the housing 20 in the vicinity of the feedthroughs 200, 201 may be established.

In another embodiment, shown in FIG. 10, only one connection terminal 202 is provided on the carrier element 24 for establishing an electrical connection to the housing 20, the connection terminal 202 being connected to the housing 20 by an electrical interconnection 204. Herein, in the shown embodiment a central connection terminal 241 centrally on the connection line 240 is provided to establish an electrical interconnection to the connection line 240 and, in this way, to the output sections 224, 225 of the collector elements 220, 221 associated with the feedthroughs 200, 201.

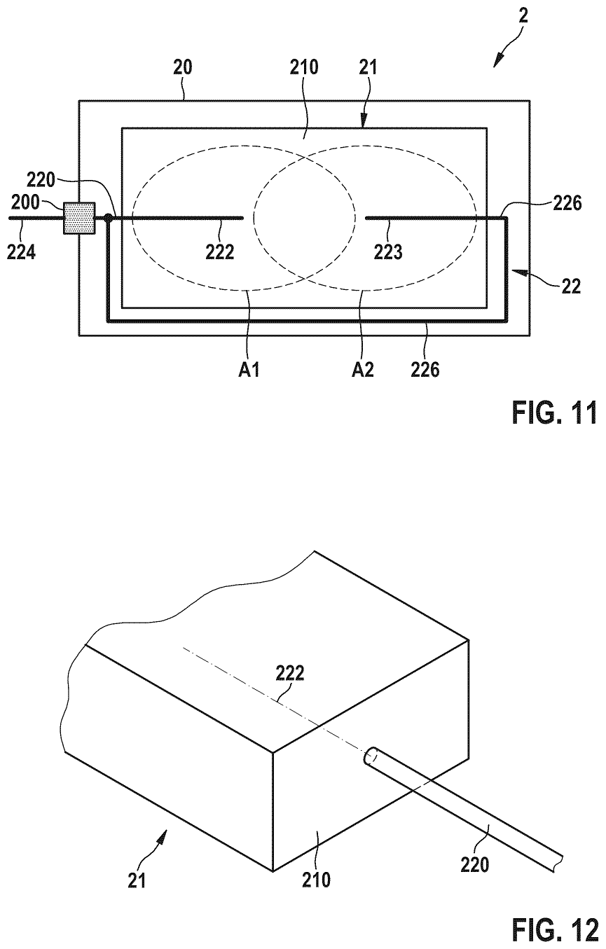

Referring now to FIG. 11, in another embodiment an electrical connection between the collector elements 220, 221 may be established by a connection line 226 received within the housing 20 of the electrical energy storage device 2. The current collector elements 220, 221 hence are electrically connected within the housing 20, but externally of the electrode element 21. In this embodiment, a single feedthrough 200 for guiding a single output section 224 through the housing 20 suffices.

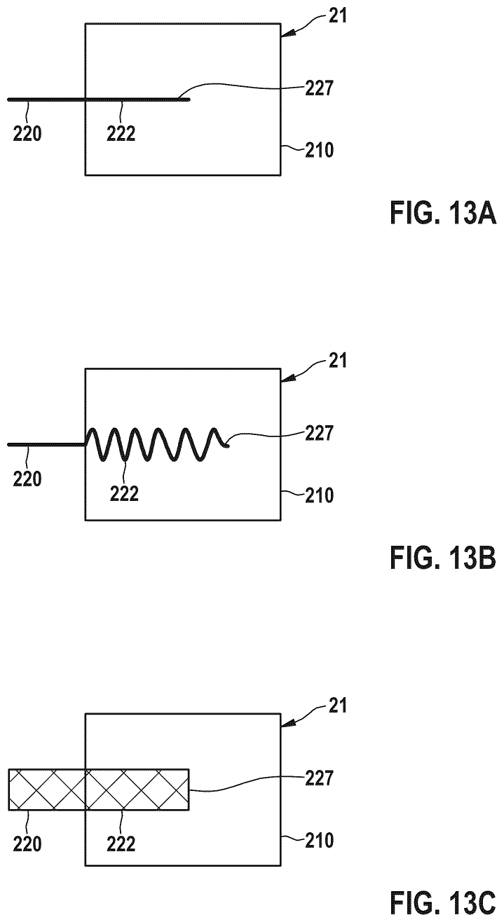

Referring now to the schematic drawings of FIGS. 13A to 13C, each current collector element 220, 221 may for example be formed by a wire or another, more complex conductive structure, for example a mesh, as described below. The current collection section 222, 223 of each current collector element 220, 221 in particular may extend straight within the electrode body 210 of the electrode element 21, as it is shown in FIG. 13A, or may follow a meandering path, as it is shown in FIG. 13B. In another embodiment, the current collection section 222, 223 may have a web or mesh shape, as shown in FIG. 13C. In each case, however, free ends 227, 228 of the current collection sections 222, 223 of the current collector elements 220, 221 are not structurally interconnected with one another within the electrode body 210 of the electrode element 21, except by the electrode body 210.

The idea of the invention is not limited to the embodiments described above, but may be implemented in an entirely different fashion.

An electrical energy storage device or an assembly of multiple electrical energy storage devices as described herein may be used in an implantable medical stimulation device, such as an implantable cardiac stimulation device, for example a stimulation device having a defibrillation function.

An electrical energy storage device or an assembly of multiple electrical energy storage devices of the type concerned herein however may also be used in other electrical or electronic devices, in particular also outside of the medical field.

### LIST OF REFERENCE NUMERALS

1 Implantable medical stimulation device
10 Lead
11 Lead
12 Generator
120 Processing circuitry
2,2A, 2B Energy storage device
20 Housing
200,201 Feedthrough
202, 203 Connection terminal
204, 205 Electrical connection
21 Electrode element
210 Electrode body
22 Current collector arrangement
220, 221 Current collector element
222, 223 Current collection section
224, 225 Output section
226 Connection line
227, 228 Free end
23 Connection arrangement
230-237 Connection line
24 Carrier element
240 Connection line
241 Connection terminal
242 Electrical connection
3 Energy storage arrangement
A1, A2 Current collection area
M Myocardium
P Plane of symmetry
RA Right atrium
RV Right ventricle
V Superior vena

1. An electrical energy storage device for an implantable medical device, comprising:
a housing,
a first electrical electrode and a second electrical electrode having different electrical polarities, one of the first electrical electrode and the second electrical electrode being formed by an electrode element having an electrically conductive electrode body arranged in the housing, and
a current collector arrangement electrically connected to the electrode element,
n the current collector arrangement comprises a first current collector element forming a first current collection section having a first free end and a second current collector element forming a second current collection section having a second free end, wherein the first current collection section and the second current collection section each extend in the electrode body or along a surface of the electrode body such that the first free end of the first collector element and the second free end of the second collector element are arranged at a distance with respect to one another in or on the electrode body.

2. The electrical energy storage device according to claim 1, wherein the first current collection section and the second current collection section are not structurally connected to each other within the volume of the electrode body, except for by the electrode body.

3. The electrical energy storage device according to claim 1, wherein the electrode body comprises a first electrical conductivity and the first current collector element and the second current collector element each comprise a second electrical conductivity, wherein the first electrical conductivity is smaller than the second electrical conductivity.

4. The electrical energy storage device according to claim 1, wherein in the first current collection section of the first current collector element and the second current collection section of the second current collector element are each embedded within the material of the electrode body.

5. The electrical energy storage device according to claim 1, wherein the first current collection section and the second current collection section extend along different directions in or on the electrode body.

6. The electrical energy storage device according to claim 1, wherein the housing is made from an electrically conductive material, wherein the other of the first electrical electrode and the second electrical electrode is formed by the housing or is electrically connected to the housing.

7. The electrical energy storage device according to claim 1, wherein the current collector arrangement comprises at least one output section, wherein the housing comprises at least one feedthrough for guiding said at least one output section through the housing.

8. The electrical energy storage device according to claim 1, wherein the housing comprises at least one rounded edge.

9. The electrical energy storage device according to claim 1, wherein the housing, the electrode element and the current collector arrangement comprising said first current collector element and said second current collector element are arranged symmetrically with respect to a plane of symmetry, the first current collector element being arranged at a first side of said plane of symmetry and the second current collector element being arranged at a second side of said plane of symmetry.

10. The electrical energy storage device according to claim 9, wherein feedthroughs of the housing for guiding output sections of the first current collector element and the second current collector element through the housing are arranged symmetrically with respect to said plane of symmetry.

11. The electrical energy storage device according to claim 1, wherein a connection line electrically connecting said first current collector element and said second current collector element, said connection line being arranged outside of the electrode body.

12. An assembly of at least two electrical energy storage devices according claim 1, wherein a connection arrangement for electrically connecting the at least two electrical energy storage devices.

13. The assembly according to claim 12, wherein the at least two electrical energy storage devices are electrically connected in series.

14. The assembly according to claim 12, wherein the housings of a first of the at least two electrical energy storage devices and of a second of the at least two electrical energy storage devices each comprise a rounded face and a planar face, wherein the first and the second of the at least two electrical energy storage devices are arranged with respect to one another such that the housings of the first and the second of the at least two energy storage devices-face towards each other with the planar faces and face away from one another with the rounded faces.

15. An implantable medical stimulation device comprising an electrical energy storage device according to claim 1.
